(19) United States
(12) Patent Application Publication
(10) Pub. No.: US 2022/0022850 A1
(43) Pub. Date: Jan. 27, 2022

(54) URINARY STONE COLLECTION DEVICE

(71) Applicant: FREESTONE, Marseille (FR)

(72) Inventors: Philippe GIAIME, Marseille (FR); Jerome SAMPOL, Marseille (FR)

(21) Appl. No.: 17/414,186

(22) PCT Filed: Dec. 10, 2019
(86) PCT No.: PCT/FR2019/000206

§ 371 Date: Jun. 15, 2021

(30) Foreign Application Priority Data
Dec. 17, 2018 (FR) 1873026

(51) Int. Cl. A61B 10/00; B01D 29/03

(52) U.S. Cl. CPC A61B 10/007; B01D 29/035

(57) ABSTRACT

A urinary stone collecting device comprising: a filter part having a concave face and an opposite convex face, and a multiplicity of holes forming a filter, a lid part connected to the filter part by a hinge, so as to be movable between a deployed position where it acts as a handle, and a closed position where it covers the concave face of the filter part and encloses a volume between the filter and lid parts, in order to retain urinary stones collected by the filter, and a locking device for the filter and lid parts in the closed position.

### CROSS-REFERENCE TO RELATED APPLICATIONS

This application is a 371 National Stage of International Application No. PCT/FR2019/000206, filed Dec. 10, 2019, which claims priority to French Patent Application No. 1873026, filed Dec. 17, 2018, the disclosures of which are herein incorporated by reference in their entirety.

### TECHNICAL FIELD

The present disclosure relates to a device for collecting urinary stones.

### BACKGROUND

Such urinary stones are the cause of renal colic. This extremely common disease, which tends to spread, is due to stones ending in the urinary tract. The result is a lack of drainage, which causes tension in the kidney cavities and very intense pain. It turns out that stone formation is more frequent in obese patients, as well as in diabetic patients.

In addition, the nature of the stones can vary from one patient to another. For example, calcium oxalate and uric acid stones are more common in men, while calcium phosphate stones are more common in women.

It also appears that the rate of recurrence of stones is very high, around 50% in France, after an average period of 3.5 years between two stone occurrences. It is therefore essential to try to avoid recurrence and therefore to prevent the occurrence of kidney and/or urinary stones. To this end, several treatment protocols exist, but their definition depends on the composition of the stones. The composition of the stones cannot be determined with certainty by means such as blood or urine analysis. It is therefore necessary to collect the stones for a direct analysis by spectrophotometry.

Currently, patients are asked to use a paper filter (e.g., coffee filter) or a cloth pad to collect urinary stones. These procedures are impractical and unhygienic, as they bring the patient's fingers into contact with the urine, especially when collecting the stone in the filter or pad. In addition, the paper filter may tear or spill during collection, resulting in the loss of the stones. These devices are for single use only. Therefore, the patient must have several filters or swabs available to make several collection attempts over a day. In practice, very few urinary stones are collected this way (less than 15%).

It also turns out that the knowledge of the pH (hydrogen potential) of the urine at the time of the collection of the stone is decisive to obtain a reliable diagnosis.

U.S. Pat. No. 2,896,788 discloses a rigid, foldable paper device in the shape of a pyramidal conduit, and comprising an internal flap forming a filter, which can be folded back to close the conduit. This device requires the patient to retrieve the stone from the filter and place it, for example, in a sealed bag, for sending to a laboratory for analysis. After use, the device is impregnated with urine and must be discarded.

U.S. Pat. No. 5,762,071 discloses a urinary stone collection device comprising a cylindrical body, in which a filter is arranged, and two covers adapted to screw onto the two opposite openings of the cylindrical body. Once closed on both sides, this device can be sent to a laboratory for analysis. This device is impractical because of the presence of three separate parts, and the need to unscrew and screw back the two covers at each collection attempt.

### SUMMARY

It is therefore desirable to provide a urinary stone collection device that is easy to use by both women and men. Such a collection device should also be able to hold the collected stones, while offering some hygiene by not requiring the patient to bring his or her fingers into contact with the urine. It may also be desirable that the collection device be usable multiple times until stones are collected, and for the collected stones to be transferable to a laboratory for analysis without the need of manipulation. It may also be desirable to offer the patient the possibility to perform a urine pH analysis.

Embodiments relate to a urinary stone collecting device comprising: a filter part having a concave face and an opposite convex face, and a multiplicity of holes forming a filter, a lid part connected to the filter part by a hinge, so as to be movable between a deployed position where it acts as a handle, and a closed position where it covers the concave face of the filter part and encloses a volume between the filter and lid parts, in order to retain urinary stones collected by the filter, and a locking device for the filter and lid parts in the closed position. According to an embodiment, the filter part comprises a receptacle for collecting urine for performing a urine pH analysis.

According to an embodiment, the hinge has a hinge axis oriented parallel to an opening plane of the filter part.

According to an embodiment, the lid part has a generally similar shape to the filter portion.

According to an embodiment, the hinge has a hinge axis oriented perpendicular to an opening plane of the filter part.

According to an embodiment, the lid part has a concave face and a convex face, the convex face of the lid being arranged in the closed position opposite the filter part, so as to ensure the locking of the filter and lid parts in the closed position.

According to an embodiment, the device comprises a flap covering the concave face of the filter part, the flap being connected by a hinge to the filter part and/or to the lid part, so as to be movable between a closed position in which it cooperates with the lid part to form a closed volume housing the filter part, and a deployed position in which it cooperates with the lid part in order to serve as a handle for holding the filter part.

According to an embodiment, the lid part comprises two side portions pivotally connected to the filter part, so as to be movable between a closed position where they form a closed volume housing the filter part, and a deployed position where they cooperate together to form a handle for holding the filter part.

According to an embodiment, the lid part forms a housing for receiving a roll of urine pH test strips.

Embodiments may also relate to a urinary stone collection assembly comprising: a collection device as previously defined, urine pH test strips, a sheet for identifying and recording a urine pH test result, and an envelope for transmitting the collection device and the identification and recording sheet.

According to an embodiment, the assembly comprises a pocket with a closure for receiving and sealing the collection device.

### BRIEF DESCRIPTION OF THE DRAWINGS

Non limitative exemplary embodiments of the disclosure will be described in the following, in relation to the attached drawings, among which:

FIGS. 1A, 1B are perspective views, respectively of the lower and upper sides of a urinary stone collection device in deployed configuration, according to an embodiment,

FIG. 2 is a side view of the collection device of FIG. 1A, in the deployed configuration,

FIG. 3 is a perspective view of the underside of the collection device of FIG. 1A in a closed configuration,

FIG. 4 is a side view of the collection device of FIG. 1A, in a closed configuration,

FIGS. 5A, 5B are perspective views, respectively of the outer and inner sides of a urinary stone collection device in deployed configuration, according to another embodiment,

FIG. 6 is a side view of the collection device of FIG. 5A, in the deployed configuration,

FIGS. 7A, 7B are perspective views, respectively of the lower and upper sides of the collection device of FIG. 5A, in closed configuration,

FIGS. 8A, 8B are perspective views, respectively of the upper and lower faces of a urinary stone collection device in deployed configuration, according to another embodiment,

FIG. 9 is a longitudinal cross-sectional view of the collection device in FIG. 8A, in the deployed configuration,

FIGS. 10A, 10B are perspective views, respectively of the lower and upper sides of the collection device of FIG. 8A, in closed configuration,

FIG. 11 is a longitudinal cross-sectional view of the collection device of FIG. 8A, in a closed configuration.

### DETAILED DESCRIPTION

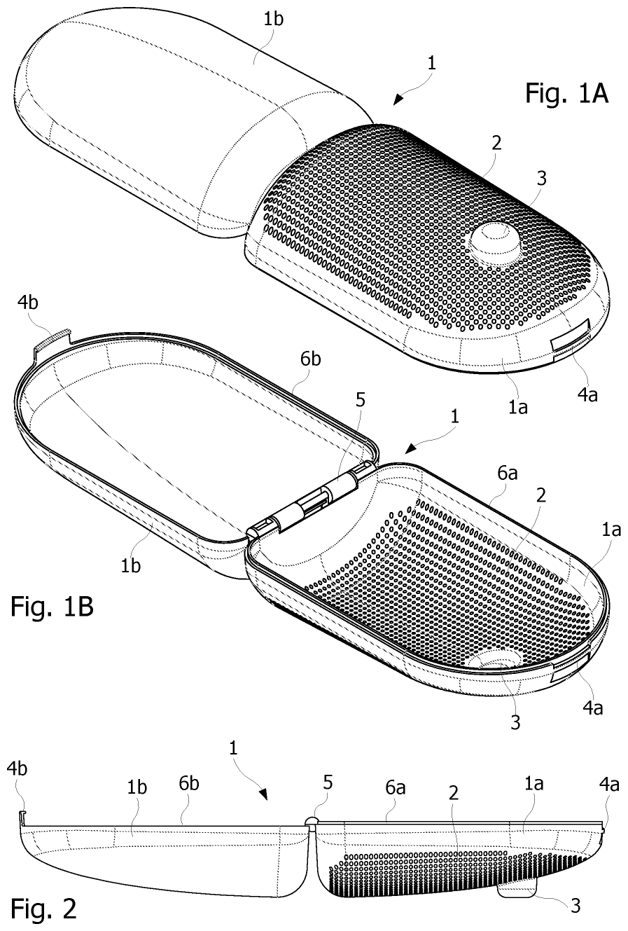

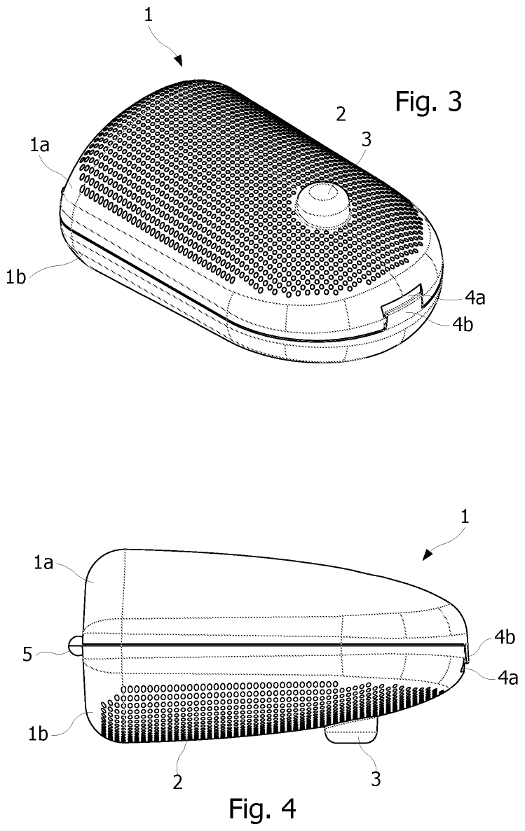

FIGS. 1A, 1B, 2, 3, 4 show a device 1 for collecting urinary stones, according to an embodiment. The device 1 comprises two half-shell-shaped parts 1a, 1b, namely a filter part 1a and a lid part 1b. Each part 1a, 1b has a concave external face and a convex internal face. The filter part 1a has a multiplicity of holes so as to form a filter 2. The width of the holes is adapted to retain urinary stones of one or more millimeters in width. The distribution and number of holes are adapted to avoid any risk of urine accumulation in the filter part. The parts 1a, 1b are connected to each other by a hinge 5, so that they can pivot between a deployed position shown in FIGS. 1A, 1B, and 2 and a closed position shown in FIGS. 3 and 4.

In the deployed position, the parts 1a, 1b may be arranged in line with each other, on either side of the hinge 5, with their respective concave sides on the same side. In this position, the lid part 1b forms a handle or grip for holding the device 1 in a position suitable for collecting urinary stones. The dimensions of the parts 1a, 1b are adapted to allow both a woman and a man to urinate through the filter 2. In the closed position, the parts 1a, 1b form a closed volume, so as to retain urinary stones collected on the filter part 1a. Thus, part 1b forms a cover adapted to the shape of the filter part 1a.

The filter part 1a has a locking device 4a for maintaining the closed position, arranged to cooperate with a complementary shaped locking device 4b provided on the lid part 1b. The hinge 5 has a pivot axis located in a plane parallel to the planes of the openings of the parts 1a, 1b. The locking devices 4a, 4b are arranged on the edges 6a, 6b of the parts 1a, 1b opposite the hinge 5. The edges 6a, 6b of the parts 1a, 1b may be arranged in such a way that in the closed position the edge (6a in the example of FIG. 1B) of one of the parts (1a) is inside the edge (6b) of the other part (1b).

The filter part 1a may have a substantially oval shape in an opening plane, with a straight edge portion on the side of the hinge 5. It may be deeper on the side of the hinge 5 than on the side of the locking device 4a. The lid part 1b may have a shape identical to the filter part 1a. However, the lid part 1b need not be as deep as the filter part 1a, since it merely serves as a handle in the extended position and as a cover in the closed position.

According to an embodiment, the filter part 1a also comprises a receptacle 3 arranged in the region of the filter 2, so that some urine can be retained, in order to allow analysis of the urinary pH, for example subsequently to the collection of urinary stones. The receptacle 3 may be arranged along a longitudinal axis of the filter part 1a, in a region between half and three quarters of the filter part starting from the axis of the hinge 5.

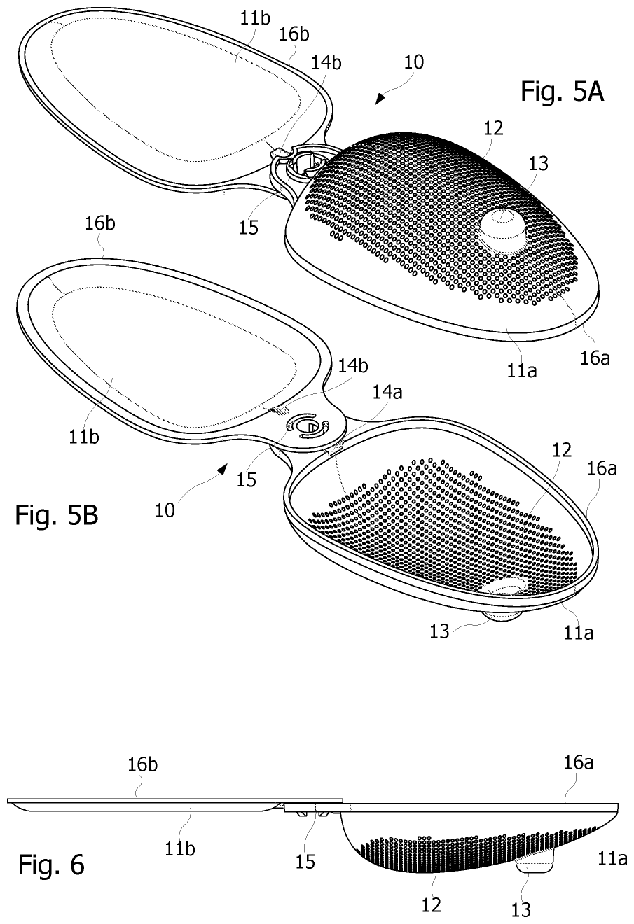

FIGS. 5A, 5B, 6, 7A, 7B show a device 10 for collecting urinary stones, according to another embodiment. The device 10 differs from the device 1 primarily in that the filter part 11a and the lid part 11b of the device 10 are connected to each other by a hinge 15 that pivots about an axis perpendicular to the plane of the edge 16a of the opening of the filter part 11a. Thus, the edges 16a, 16b of the filter part 11a and lid part 11b remain in the same plane during their relative movements between the closed and deployed positions. The lid part 11b is substantially planar or has a slightly convex inner face and a corresponding slightly concave outer face, to engage the filter part 11a in the closed position.

The filter part 11a comprises a multiplicity of holes distributed to form a filter 12, and may comprise a receptacle 13 for retaining a small amount of urine to perform a urine pH test.

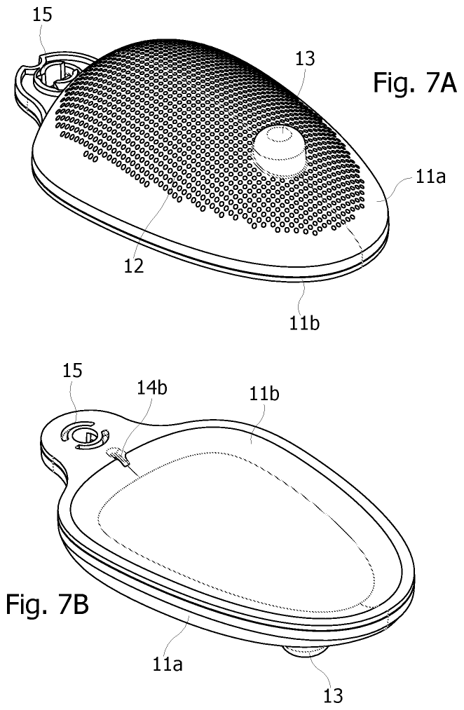

To hold the device 10 in the closed configuration, a detent system is arranged on facing edge portions of the parts 11a, 11b. The detent system comprises in the vicinity of the hinge 15 a bump 14b arranged on one of the parts 11a, 11b to engage in the closed position in a notch 14a arranged on the other part 11a, 11b. In the example of FIGS. 5A, 5B, the bump 14b is formed in the lid part 11b and the notch 14b is formed in the filter part 11a. It should be noted that the detent system 14a, 14b may be omitted, particularly if the lid part 11b has a convex face that engages the filter part 11a in the closed position, as is apparent in particular in FIGS. 6 and 7B.

In an embodiment, it is intended to completely enclose the filter part, so that, in particular after contact with urine, it can be completely isolated in a closed volume. To this end, the device comprises a lower part mounted so as to be movable between a closed position in which it covers the lower convex (external) face of the filter part and locks with the latter or with the lid part which covers the upper concave (internal) face of the filter part, and a deployed position in which it cooperates with the lid part to form a handle or a grip enabling the device to be held during the collection of urinary stones.

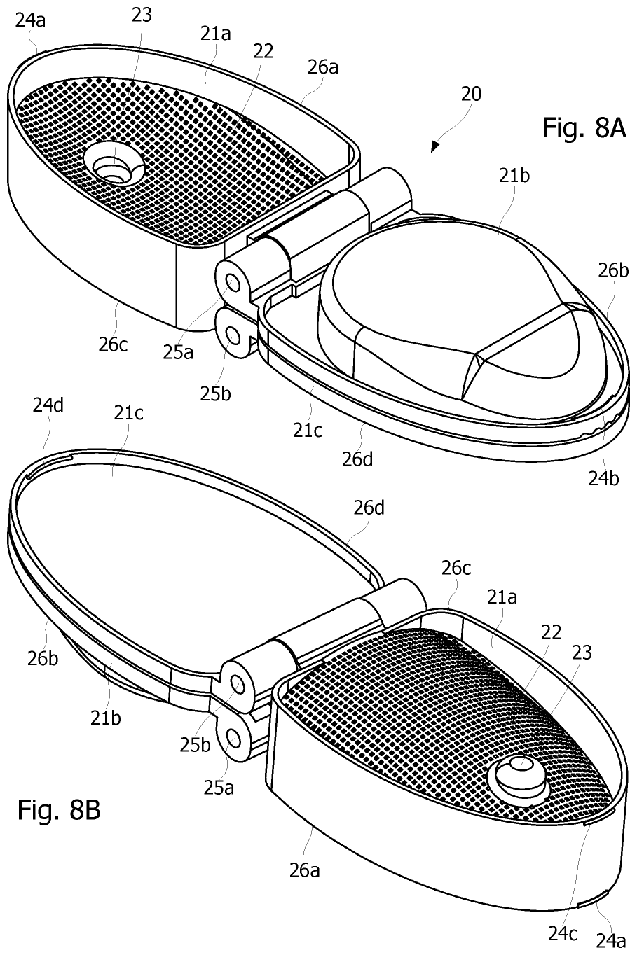

FIGS. 8A, 8B, 9, 10A, 10B, 11 show a device 20 for collecting urinary stones, according to another embodiment. The device 20 differs from the device 1 mainly in that it comprises a lower flap 21c configured to cover the lower face of a filter 22 and to lock in a closed position on a filter part 21a, the lower flap 21c being linked to the filter part 21a by a hinge 25b. The filter part 21a has a tubular shape open at its two ends delimited by upper and lower edges 26a, 26c. The passage between the two ends of the filter part 21a is closed by the curved filter 22, having a concave upper face and a convex lower face. The filter 22 may include a receptacle 23 for retaining a small amount of urine to perform a urine pH test. The device 20 comprises a lid part 21b linked to the filter part 21a by a hinge 25a.

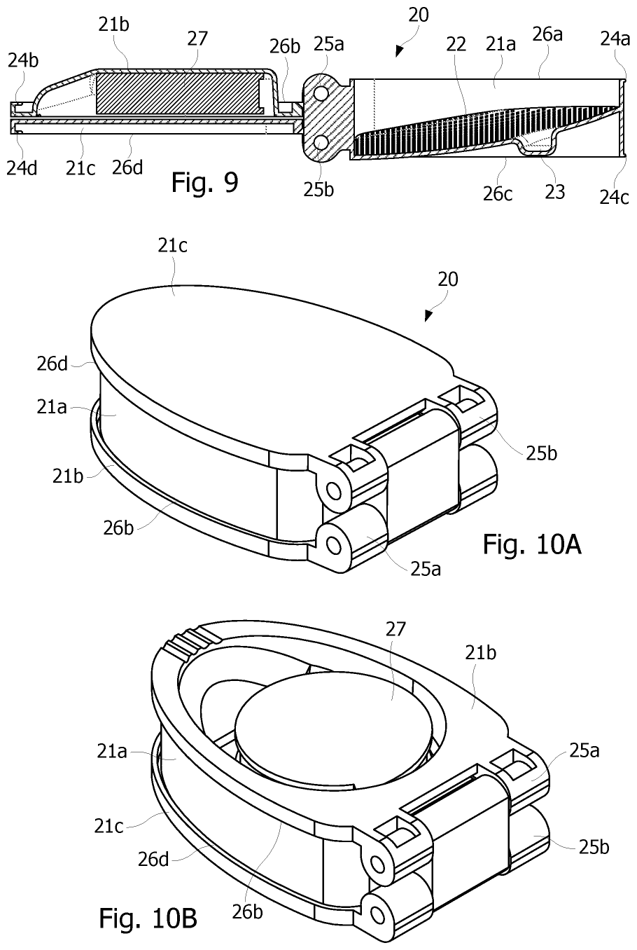

The hinges 25a, 25b are configured so that in the deployed configuration (FIGS. 8A, 8B, 9), the lid part 21a and the flap 21c can be placed against each other so that they can form a handle or grip suitable for holding with one hand. In the example of FIGS. 8A, 8B, 9, 10A, 10B, 11, the axes of the hinges 25a, 25b extend in planes parallel to the planes of the edges 26a, 26c of the filter part 21a.

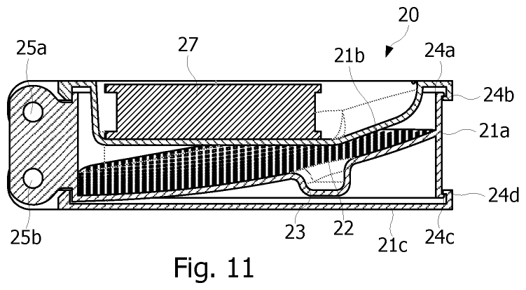

The filter part 21a has locking devices 24a, 24c for maintaining the closed position, arranged to cooperate respectively with complementary shaped locking devices 24b, 24d provided respectively on the edge 26b of the lid part 21b and on the edge 26d of the flap 21c. The locking devices 24a-24d are arranged in a region of the edges 26a-26d of the parts 21a, 21b and 21c opposite the hinges 25a, 26b. The edges 26a-26d of the parts 21a, 21b, 21c can be arranged so that in the closed position, edges (26a, 26c in particular in FIG. 11) are inside the facing edges (26b, 26d).

According to an embodiment, the lid part 21a has an outer face (upper face in the closed position of the lid part) forming a housing provided to receive a roll 27 of urine pH test strips (FIGS. 9, 10B, 11).

According to an embodiment, the device 1, 10, 20 is entirely made by molding or 3D printing in a plastic material. It has a length between 8 and 12 cm, a width between 5 and 7 cm, and depth between 1.5 and 5 cm. The holes forming the filter 2, 12, 22 have a width between 0.5 and 1 mm. The receptacle 3, 13, 23 may have a width and a depth between 0.8 and 1.5 cm.

The device 1, 10, 20 may be distributed in combination with a waterproof bag in which the patient can place the device 1 after use, urine pH test strips that can be used with the urine retained in the receptacle 3, and a card on which to write identification information and the result of the urine pH test.

The hinge 5, 25a, 25b may be realized in different ways. For instance, in the case of a hinge parallel to the plane of the opening of the filter part, the hinge may simply be made with a flexible membrane integral with parts 1a and 1b (or 21a and 21b, or 21a and 21c), for example in the same material molded in a single piece including both parts. In this case, the device 1 (or the two parts 21a, 21b or 21a, 21c) can be made in one piece.

According to another embodiment, the hinge 5, 25a, 25b is realized in the form of a cylindrical shaft integral with one of the two parts 1a and 1b (or 21a and 21b, and/or 21a and 21c), pivoting in grooves of cylindrical form, integral with the other of the two parts.

It will be apparent to a person skilled in the art that the present disclosure is susceptible to various alternative embodiments. In particular, the disclosure is not limited to the previously described embodiments shown in the figures. For instance, in all the previously described embodiments, the axis of articulation may be parallel or perpendicular to the plane of the opening of the filter part. When the hinge axis is perpendicular to the plane of the opening of the filter part, as in the example shown in FIGS. 5A, 5B, it can be envisaged to play on the flexibility of the material forming the device to make the concave face of the lid part 11b (and/or of the flap 21c) pass between its closed and deployed positions.

According to another embodiment, the lid part is made of two lateral parts, for example in the shape of half-shells, pivotally fixed to the filter part along an axis perpendicular to the plane of the opening of the filter part, so as to be movable between a closed position in which they form a closed volume including the filter part, and a deployed position in which they form together a handle for holding the filter part.

Alternatively, the filter 2, 12, 22 can be made in the form of holes aligned in rows and/or columns, or in the form of a grid formed by two sets of parallel ribs, with the ribs of one set having different orientations than the ribs of the other set.

It may also be observed that the edges of the filter and lid parts are not necessarily flat. Indeed, they have complementary shapes to ensure a complete closure of the volume delimited by the filter and lid parts, in order to retain the stones that can be collected by the filter part.

1. A urinary stone collecting device comprising:
a filter part having a concave face and an opposite convex face, and a multiplicity of holes forming a filter,
a lid part connected to the filter part by a hinge, so as to be movable between a deployed position where it acts as a handle, and a closed position where it covers the concave face of the filter part and encloses a volume between the filter and lid parts, in order to retain urinary stones collected by the filter, and
a locking device configured to lock the filter and lid parts in the closed position,
wherein the filter part comprises a receptacle for collecting urine for performing a urine pH analysis.

2. The device of claim 1, wherein the hinge has a hinge axis oriented parallel to an opening plane of the filter part.

3. The device of claim 1, wherein the lid part has a generally similar shape to the filter portion.

4. The device of claim 1, wherein the hinge has a hinge axis oriented perpendicular to an opening plane of the filter part.

5. The device according to claim 4, wherein the lid part has a concave face and a convex face, the convex face of the lid part being arranged in the closed position opposite the filter part, so as to ensure the locking of the filter and lid parts in the closed position.

6. The device according to claim 1, comprising a flap covering the concave face of the filter part, the flap being connected by a hinge to the filter part and/or to the lid part, so as to be movable between a closed position in which it cooperates with the lid part to form a closed volume housing the filter part, and a deployed position in which it cooperates with the lid part in order to serve as a handle for holding the filter part.

7. The device according to claim 1, wherein the lid part comprises two side portions pivotally connected to the filter part, so as to be movable between a closed position where they form a closed volume housing the filter part, and an deployed position where they cooperate together to form a handle for holding the filter part.

8. The device according to claim 1, wherein the lid part forms a housing for receiving a roll of urine pH test strips.

9. A urinary stone collection assembly comprising:
a collection device, comprising:
a filter part having a concave face and an opposite convex face, and a multiplicity of holes forming a filter,
a lid part connected to the filter part by a hinge, so as to be movable between a deployed position where it acts as a handle, and a closed position where it covers the concave face of the filter part and encloses a volume between the filter and lid parts, in order to retain urinary stones collected by the filter, and
a locking device configured to lock the filter and lid parts in the closed position,
wherein the filter part comprises a receptacle for collecting urine for performing a urine pH analysis,
urine pH test strips,
a sheet for identifying and recording a urine pH test result, and
an envelope for transmitting the collection device and the identification and recording sheet.

10. The assembly of claim 9, comprising a pocket with a closure for receiving and sealing the collection device.

11. The assembly of claim 9, wherein the hinge has a hinge axis oriented parallel to an opening plane of the filter part.

12. The assembly of claim 9, wherein the lid part has a generally similar shape to the filter portion.

13. The assembly of claim 9, wherein the hinge has a hinge axis oriented perpendicular to an opening plane of the filter part.

14. The assembly of claim 13, wherein the lid part has a concave face and a convex face, the convex face of the lid part being arranged in the closed position opposite the filter part, so as to ensure the locking of the filter and lid parts in the closed position.

15. The assembly of claim 9, comprising a flap covering the concave face of the filter part, the flap being connected by a hinge to the filter part and/or to the lid part, so as to be movable between a closed position in which it cooperates with the lid part to form a closed volume housing the filter part, and a deployed position in which it cooperates with the lid part in order to serve as a handle for holding the filter part.

16. The assembly of claim 9, wherein the lid part comprises two side portions pivotally connected to the filter part, so as to be movable between a closed position where they form a closed volume housing the filter part, and an deployed position where they cooperate together to form a handle for holding the filter part.

17. The assembly of claim 9, wherein the lid part forms a housing for receiving a roll of urine pH test strips.
